(19) United States
(12) Patent Application Publication
(10) Pub. No.: US 2011/0144517 A1
(43) Pub. Date: Jun. 16, 2011

(54) Video Based Automated Detection of Respiratory Events

(76) Inventor: Miguel Angel Cervantes, Barcelona (ES)

(21) Appl. No.: 13/032,867

(22) Filed: Feb. 23, 2011

Related U.S. Application Data
(63) Continuation-in-part of application No. 12/321,840, filed on Jan. 26, 2009.

(51) Int. Cl. A61B 5/087

(52) U.S. Cl. 600/538

(57) ABSTRACT

A computer implemented method for automated sleep monitoring, including recording live images of a subject sleeping, transmitting the recorded images to a computing device, receiving the transmitted images at the computing device, performing motion analysis of the subject based on the received images, computing an air flow signal that represents the amount of air flowing into the lungs of the subject over time from the results of said motion analysis, automatically detecting respiratory events based on said airflow signal, wherein a respiratory event is a breathing disturbance, and displaying the respiratory events experienced by the subject on a monitor. An apparatus is also described.

### PRIORITY CLAIM

This is a Continuation-in-Part of patent application Ser. No. 12/321,840 filed Dec. 29, 2005 by inventor Miguel Angel Cervantes which should be incorporated by reference in the present application.

### FIELD OF THE INVENTION

The present invention relates to digital image and video processing and automated detection of respiratory events while sleeping.

### BACKGROUND OF THE INVENTION

The present invention concerns the analysis of respiration and the detection of respiratory events from a series of digital images such as those provided by a digital video camera. The basis for such analysis is that the volume of air that circulates in the lungs is proportional to the movement that a person, also referred to herein as a subject, makes as a consequence of breathing.

The present invention concerns respiratory events, such as apneas and hypopneas, also referred to as hypo-apneas, in which breathing is disturbed during sleep for a limited period of time. Typically, the beginning of an apnea is characterized by a decrease of airflow to the lungs and the end of an apnea is characterized by a large inflow of air into the lungs when the respiratory tract unlocks and breathing recommences. Visually, at the beginning of an apnea there is a decrease in the movement in the muscles of the chest that restricts the movement of the chest during normal respiration and at the end of the apnea there is an expansion of the chest.

Use of video images to analyze posture changes and respiration rates of subjects is described in Nakajima, K., Matsumoto, Y. and Tamura, T., “Development of real-time image sequence analysis for evaluating posture change and respiratory rate of a subject in bed”, Physiol. Meas. 22 (2001), pgs. N21-N28. Nakajima et al. describe generating a waveform from captured video images, that approximates true respiratory behavior. Nakajima et al. use optical flow measurements to estimate motion velocities. They describe real-time generation of waveforms of average velocities. They also relate visual patterns within average velocity waveforms to states including “respiration”, “cessation of breath”, “full posture change”, “limb movement”, and “out of view”.

Although Nakajima et al. identify states manually from visual inspection of their waveforms, they do not disclose automated detection of respiratory events or estimation of breathing motion.

A method and apparatus for determining, monitoring and predicting levels of alertness by detecting microsleep episodes is described in U.S. Pat. No. 6,070,098, filed Apr. 10, 1998, to Moore-Ede, et al. Moore-Ede describes the operation and function of what is commonly referred to as a polysomnography system or device, i.e. an apparatus that performs polysomnography. Its analysis relies on data from multiple sensor channels. Moore-Ede uses video data to identify certain fatigue-related events such as yawning or head snapping. It does not teach the use of image or video analysis to estimate breathing motion or to automatically detect respiratory events.

Generally, polysomnography systems and devices are typically capable of monitoring respiratory airflow. However, such monitoring is typically performed by measuring temperature as a surrogate for airflow or by directing measuring nasal airflow. Analysis of digital images or digital video has not been used in polysomnography systems and devices to estimate breathing motion or to automatically detect respiratory events without supplemental information coming from other sensor channels.

### SUMMARY OF THE DESCRIPTION

The present invention concerns apparatus and methods for automated detection of respiratory events during sleep, using image processing to continuously monitor movements of a person during sleep. The apparatus of the present invention preferably includes two units, a live video recorder mounted near the subject being monitored and a computing device that is generally remote from the video recorder. The present invention uses a video recorder, which is not in direct contact with the subject being monitored.

The present invention uses motion analysis to identify a time history of a subject's movements. The motion information is stored for post-analysis review and diagnosis. In one embodiment, the motion information is analyzed to generate an air flow signal that corresponds to the volume of respiration of a subject. The air flow signal is then processed and respiratory events are detected.

A first preferred embodiment of the present invention uses an architecture wherein computing device performs the image processing state detection, and respiratory event detection, and the video recorder can be a simple inexpensive recording device. A second embodiment of the present invention uses an architecture wherein the video recorder performs the image processing and state detection, and the computing device can be an inexpensive device with a display monitor for viewing the results. A third embodiment of the present invention uses an architecture wherein a first computing device receives images from a video recorder, performs certain elements of image processing, sends the intermediate results data to a second computing device which performs the final image processing, produces reports and provides the reports to the subject and/or third parties such as doctors, or sleep technicians across a network.

One embodiment of the subject invention is directed towards an apparatus for automatically monitoring sleep, including a video recorder for recording live images of a subject sleeping, including a transmitter for transmitting the recorded images to a computing device, and a computing device communicating with said video recorder transmitter, including a receiver for receiving the transmitted images, a motion analyzer for performing motion analysis of the subject based on the received images, a respiratory event detector for (1) computing an air flow signal that represents the amount of air flowing into the lungs of the subject over time from the results of said motion analysis, and (2) automatically detecting respiratory events based on said airflow signal, wherein a respiratory event is a breathing disturbance, and a monitor for displaying the respiratory events experienced by the subject, detected by said computing device.

Another embodiment is directed towards a computer implemented method for automated sleep monitoring, including recording live images of a subject sleeping, transmitting the recorded images to a computing device, receiving the transmitted images at the computing device, performing motion analysis of the subject based on the received images, computing an air flow signal that represents the amount of air flowing into the lungs of the subject over time from the results of said motion analysis, automatically detecting respiratory events based on said airflow signal, wherein a respiratory event is a breathing disturbance, and displaying the respiratory events experienced by the subject on a monitor.

### BRIEF DESCRIPTION OF THE DRAWINGS

The present invention will be more fully understood and appreciated from the following detailed description, taken in conjunction with the drawings in which:

FIG. 1 is a simplified block diagram of a real-time mobile sleep monitoring system according to a first, non-embedded, architecture, wherein state and respiration analysis are performed by a computing device, in accordance with a preferred embodiment of the present invention;

FIG. 2 is a simplified block diagram of a version of the monitoring system of FIG. 1 whereby the computing device is a personal computer that is connected to a mobile display unit, in accordance with a preferred embodiment of the present invention;

FIG. 3 is a simplified block diagram of a real-time sleep monitoring system according to a second, embedded architecture, wherein state and respiration analysis are performed by a video capture device, in accordance with a preferred embodiment of the present invention;

FIG. 4 is a simplified flowchart for a method of monitoring sleep by a computing device in real-time according to a first architecture, wherein state and respiration analysis are performed by the computing device, in accordance with a preferred embodiment of the present invention;

FIG. 5 is a simplified flowchart for a method of monitoring sleep by a computing device in real-time according to a second architecture, wherein state and respiration analysis are performed by a video capture device, in accordance with a preferred embodiment of the present invention;

FIG. 6 is a simplified block diagram of a high-sensitivity motion analyzer, in accordance with a preferred embodiment of the present invention;

FIG. 7 is a simplified block diagram of a state detector, in accordance with a preferred embodiment of the present invention;

FIG. 8 is a simplified block diagram of a respiratory event detector, in accordance with a preferred embodiment of the present invention;

FIG. 9 is a simplified flow diagram of a method for detecting respiratory events, in accordance with a preferred embodiment of the present invention;

FIG. 10 is an illustration of a user interface for monitoring and analyzing respiration and respiratory events, in accordance with a preferred embodiment of the present invention;

FIG. 11 is an illustration of a user interface for monitoring and analyzing respiration and respiratory events, in accordance with a preferred embodiment of the present invention;

FIG. 12 is a simplified block diagram of a real-time sleep monitoring system according to a third, wherein motion analysis is performed by a first computing device and state detection and respiratory event detection are performed by a second computing device, in accordance with a preferred embodiment of the present invention; and

FIG. 13 is a simplified flowchart for a method of monitoring sleep according to a third architecture, wherein motion analysis is performed by a first computing device and state detection and respiratory event detection are performed by a second computing device, in accordance with a preferred embodiment of the present invention.

### DETAILED DESCRIPTION

The present invention concerns an apparatus and method for automated real-time sleep monitoring, based on analysis of live recorded video of a subject sleeping. The present invention has application to devices that enable physicians to monitor patients for sleep disorders such as apnea. The present application also has application to self-monitoring, for subjects who wish to monitor their own sleep behavior and be alerted when various states of alert and respiratory events are detected, and to be able to post-diagnose their sleep behavior with statistical analysis. Similar in spirit to devices that enable people to monitor their pulse as a general health indicator, devices of the present invention enable people to monitor their sleep as a healthy sleep indicator.

Overall, in a preferred embodiment, the present invention includes two units; namely, a live video recorder that is mounted in the vicinity of the subject being monitored, and a computing device also referred to as a monitoring unit. Generally, these two units are remote from one another, and the video recorder preferably communicates with the monitoring unit via wireless communication. However, in self-monitoring applications, where a subject is monitoring his own sleep, the units can be combined functionally into a single unit.

In another embodiment, the live video recorder may connect to an intermediary computing device such as a computing device or tablet computer that performs a portion of the computing and then communicates data to a second computing device that completes the processing.

As used herein the following terms have the meaning given below:

Subject refers to a person being monitored by the present invention.

Respiratory event refers to a breathing disturbance that occurs during a subject's sleep. Such an event may be classified as an apnea, hypopnea, or other breathing impairment. Typically a respiratory event is characterized by a cessation or marked reduction of nasal respiratory airflow for a period of time.

Video recorder refers to a device that records or captures a sequence of digital images or digital video frames and communicates them across an interface such as USB, USB 2, or IEEE 1394, or across a network to another device or system. A video recorder may inter alia refer to a commercial video recorder, a video camera, a digital camera, a web cam or a mobile phone or smart phone equipped with a digital recording capability. The term “live video recorder” refers to a video recorder that communicates the sequence of captured digital images or video frames in real-time.

The present invention performs real-time high-sensitivity motion detection on the images that are recorded by the video recorder, as described hereinbelow, and automatically infers state information about the subject being monitored, without manual intervention. Motion information is stored for later analysis. Such later analysis, or post-analysis, includes the automated detection of respiratory events.

Automated motion detection of the present invention is able to precisely filter out noise, and thereby accurately estimate even subtle motions of a subject. As such, the present invention applies even in situations where the subject is covered with layers of sheets and blankets.

Automated state inference of the present invention relies on several indicators. One such indicator is repetitiveness of detected movements. Empirical studies have shown that a repetitiveness pattern of a subject's movements while the subject is sleeping is very different than a repetitiveness pattern of a subject's movements while the subject is awake. Similarly, a repetitiveness pattern of movement is different for soft sleep than it is for deep sleep.

In accordance with a preferred embodiment of the present invention, repetitiveness is used as a characteristic of a subject's sleep. For example, when a subject moves, his repetitiveness pattern is broken for a short amount of time. Similarly, regarding the various stages of sleep, during the rapid eye movement (REM) stage and the stage preceding REM sleep, a subject is generally in a semi-paralyzed state where his body is paralyzed with the exception of vital muscles involved in the breathing process. Such features of sleep, combined with analysis of the subject's movements during sleep, enable the present invention to determine a likelihood that the subject is in a given stage at any given time. Thus if the subject is moving, which is manifested in a lack of repetitiveness, then he is more likely to be in a soft sleep; whereas if the subject does not move for a specific amount of time, which is manifested in a presence of repetitiveness, then he is more likely to be in a deep sleep.

Apparatus of the present invention preferably also maintains a time history log of state information, motion data, detected respiratory events, and digitized images, for post-analysis study and diagnosis. For example, a subject using the present invention to self-monitor his sleep may use such a time history of state data, such as the percentage of time a subject's total sleep is in deep sleep over a specified time period such as one week or one month, for deriving statistical measures of the quality of his sleep. Further the motion information is subsequently used to detect respiratory events.

The time history log preferably includes inter alia (i) a summary of the subject's last night's sleep, including times when the subject awoke or otherwise changed states and detected respiratory events; (ii) average of the subject's last week's or last month's sleep statistics, or such other time period; and (iii) a comparison of the subject's last night's sleep to that of the last week or month, or such other time period. Preferably, a user of the present invention presses one or more buttons to enable a display of such log information.

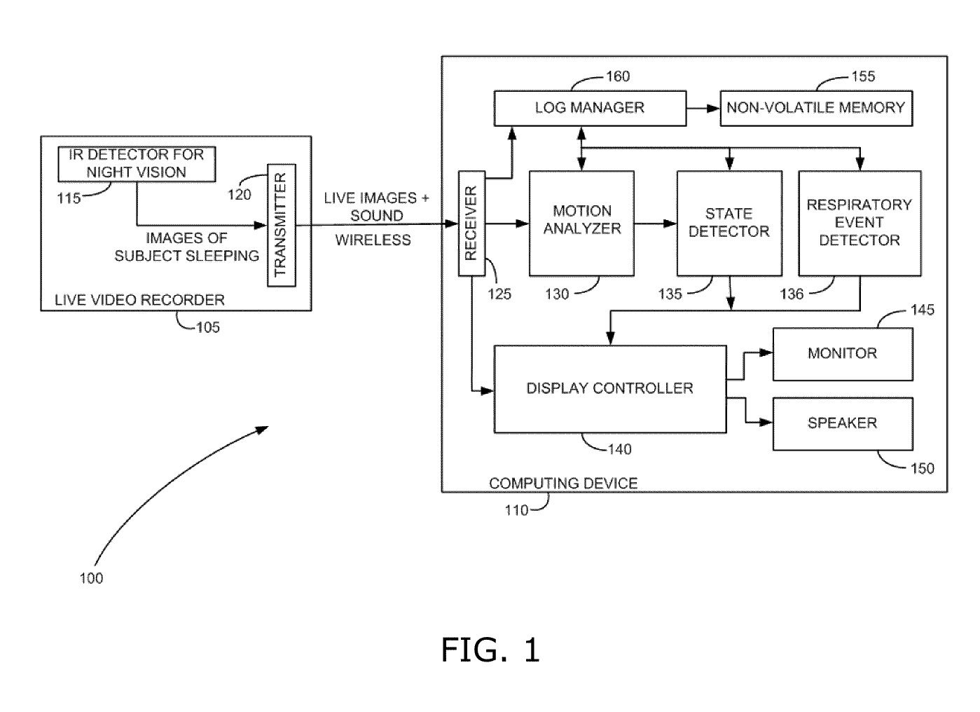

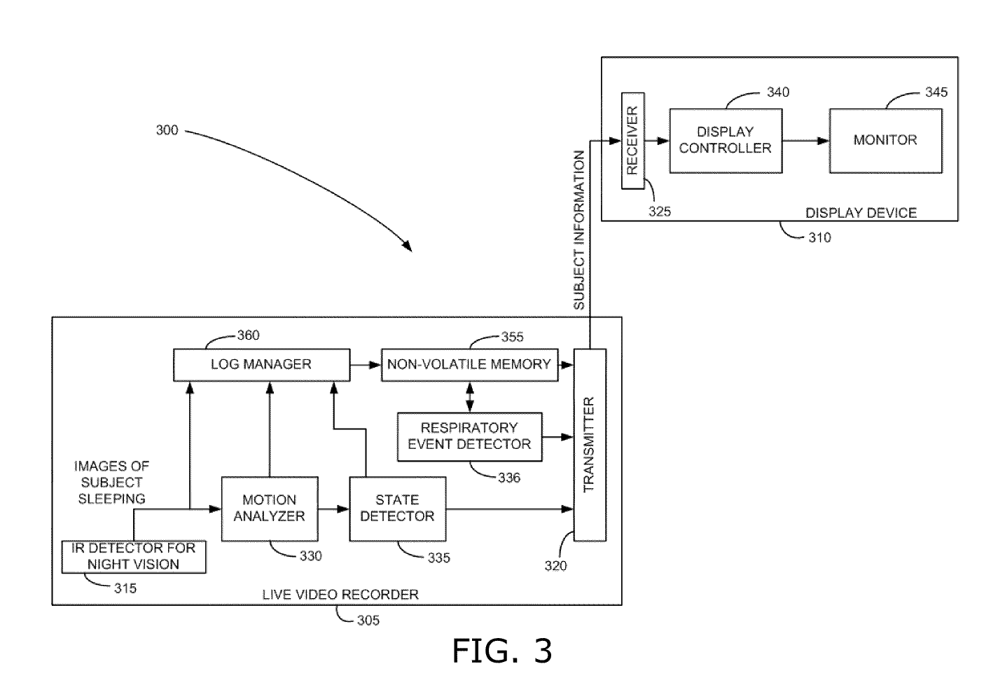

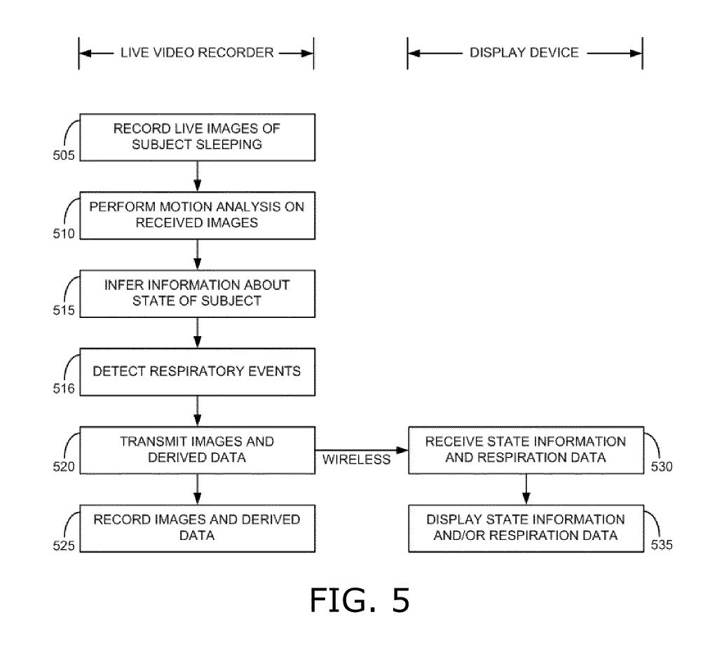

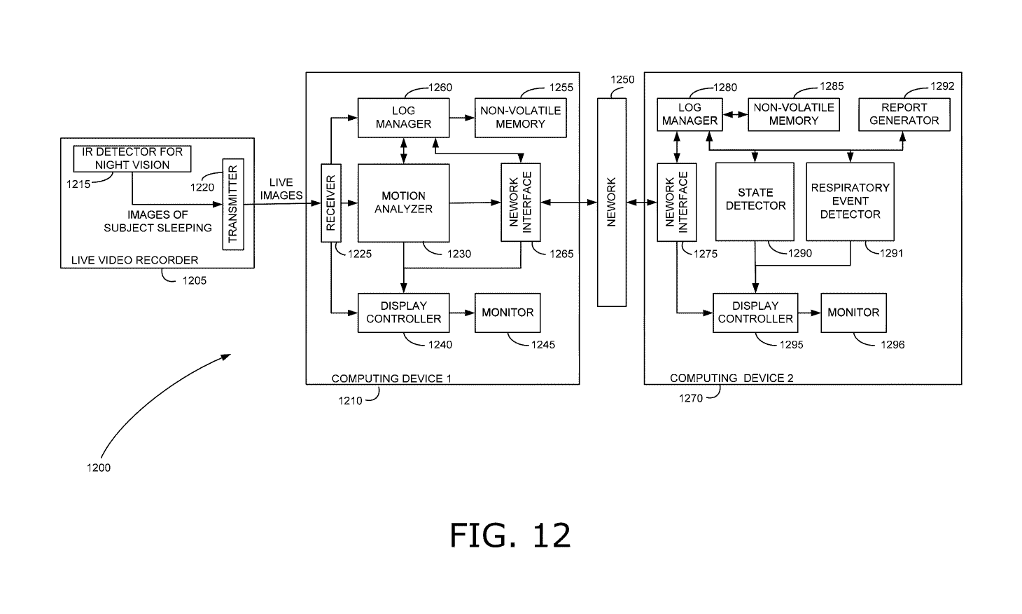

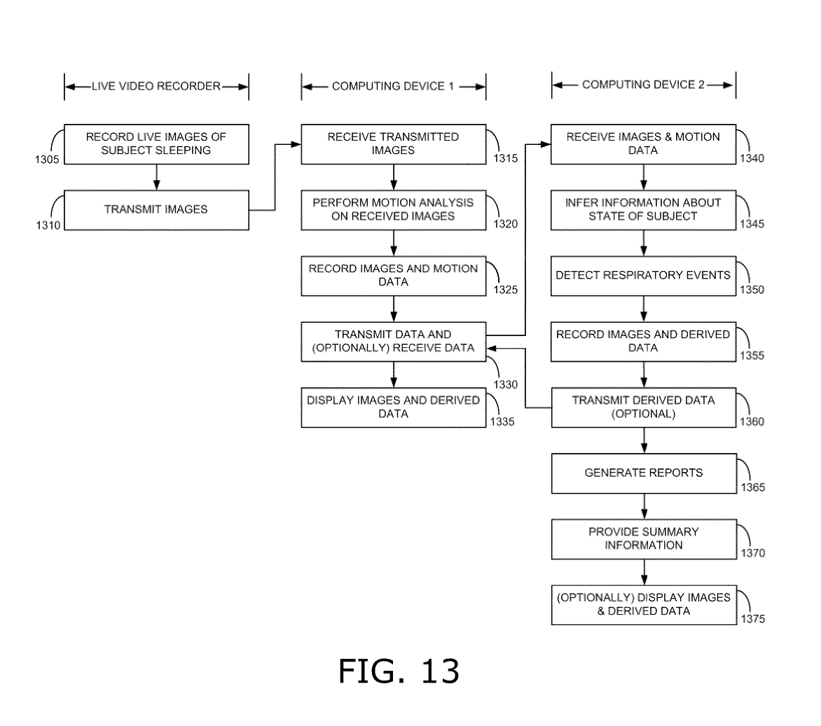

The present invention has three general embodiments; namely, a first embodiment (FIGS. 1,2 and 4 below) in which the image processing required to infer state information and detect respiratory events is performed at the monitoring unit, a second embodiment (FIGS. 3 and 5 below) in which the image processing is performed at the recording unit and a third embodiment (FIGS. 12 and 13 below) in which the image processing is allocated between two computing devices. Each embodiment has advantages over the other relating to hardware complexity, cost and interoperability.

Reference is now made to FIG. 1, which is a simplified block diagram of a real-time mobile sleep monitoring system according to a first, non-embedded, architecture, wherein state and respiration analysis are performed by a computing device, in accordance with a preferred embodiment of the present invention. Shown in FIG. 1 is an overall system including a live video recorder 105, which records images of a subject while the subject is sleeping, and a computing device 110, which processes the recorded images, infers real-time information about the state of the subject, and performs respiratory event detection. Computing device 110 may be a personal computer, a computer server, a laptop computer or other processor-based system. In addition, computing device 110 may be implemented as a network-based service performed by one or more computers that connects to video recorder 105 across a network. Preferably, video recorder 105 is mounted on a wall or on a bed or on another piece of furniture in the vicinity of the subject, in such a way that video recorder 105 can capture accurate images of the subject while he is sleeping. For example, if the subject is an person who is sleeping in a bed, then video recorder 105 may be mounted on the bed or on the wall above the bed, and directed at the sleeping person.

Preferably, a particular feature of video recorder 105 is the ability to take clear images in a dark surrounding, since this is the typical surrounding in which subjects sleep. To this end, video recorder 105 preferably includes an infrared (IR) detector 115 or such other heat sensitive or light sensitive component used to enhance night vision.

In accordance with a preferred embodiment of the present invention, computing device 110 is used to monitor the subject at a site that is remote from the location of video recorder 105. For example, if the subject is sleeping in a bed as above, then computing device 110 may be located in another room or an office.

Video recorder 105 includes a transmitter 120, which transmits the recorded images in real-time to a receiver 125 within computing device 110. In a preferred embodiment, transmitter 120 transmits the recorded images using wireless communication, so that no physical wires are required to connect video recorder 105 with computing device 110. In another embodiment, transmitter 120 communicates across a wired network or a wide area network such as the Internet with computing device 110

As receiver 125 receives the transmitted images, the images are passed to a motion analyzer 130, which performs high sensitivity motion detection as described in detail hereinbelow. The results of motion analyzer 130 are passed to a state detector 135, which infers a state of the subject, as described in detail hereinbelow. Detected states may include inter alia “sleeping”, “awake”, “light sleep”, and “deep sleep”.

State information inferred by state detector 135 is passed to a display controller 140 and may be stored in a non-volatile memory 155 which is managed by a log manager 160.

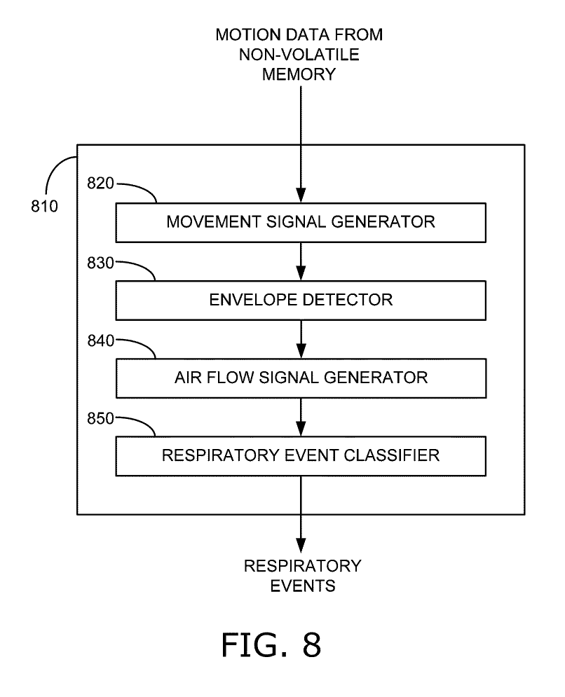

A respiratory event detector 136 further processes motion information stored in non-volatile memory 155 to detect respiratory events. The further processed motion information and detected respiratory events are stored in non-volatile memory 155 or may be displayed via display controller 140. The operation of respiratory event detector 136 is described in further detail below with reference to FIG. 8.

Log manager 160 can be used for storing in non-volatile memory 155 a time history of images, and derived information such as state information, motion information, detected respiratory events and other information that describes the subject's sleep. Such a history can be used for post-analysis, including statistical analysis of sleep patterns and interference.

Non-volatile memory 155 may include virtually any mechanism usable for storing and managing data, including but not limited to a file, a folder, a document, one or more database file(s), and the like. Non-volatile memory 155 may further represent a plurality of different data stores. For example, data storage may represent an image or video database that stores captured digital images and/or digital videos, a motion information database, a state information database, and a respiratory event database. Further, non-volatile memory 155 may also include network storage or cloud storage in which the physical storage media is accessed across a network.

Images received from receiver 125, or stored in non-volatile memory 155, state information inferred by state detector 135, and motion information including detected respiratory events may be passed to a display controller 140 for display on monitor 145. As shown in FIG. 1, display controller 140 controls both a monitor 145 and, optionally, a speaker 150. It may be appreciated by those skilled in the art that display controller 140 may include two separate device controllers, a first device controller for displaying data on monitor 145 and a second device controller for playing sound on speaker 150. It may further be appreciated that monitor 145 and speakers 150 may be integrated into computing device 110 or may be separate, connected, devices.

If video recorder 105 also records sound, then the recorded sound may also be transmitted from transmitter 120 to receiver 125, and display controller 140 may also be used for playing the sound on speaker 150. The capability for computing device 110 to display the recorded images and to play the recorded sound may be excluded from the hardware, or alternatively may be enabled in the hardware and selectively activated by a user of computing device 110.

It may be appreciated that there are various display options each of which may be suitable for a particular scenario, such as any combination of: (i) continuous or selective display of video, (ii) continuous or selective sound play, and (iii) continuous or selective state display. Selective display preferably occurs when an alert state is inferred, where an alert state is a state deemed to be significant. Specifically, the apparatus of the present invention may use settings for various modes, as described in Table I.

TABLE 1
Monitoring Settings

| Mode | Setting |
|---|---|
| Daytime | Continuous display of state information; continuous |
| monitoring | display of images; continuous sound play; sound of alarm |
| mode | when a state of alert is inferred |
| Nighttime | Selective display of state information and sound of alarm |
| monitoring | when a state of alert is inferred; selective display of |
| mode | images; selective sound play |
| Self- | Selective display of information, including: state |
| monitoring | information; log history of images and states; and |
| mode | respiration data including detected respiratory events |

It will be appreciated by those skilled in the art that different combinations of settings than those listed in Table 1 may be used instead for the various modes.

The architecture of FIG. 1 delegates the work of motion analysis, state detection, and respiratory event detection to computing device 110. An advantage of this architecture is that a conventional off-the-shelf video recorder that has wireless transmission capability can be used for video recorder 105. Thus computing device 110 is interoperable with a wide variety of video recorders.

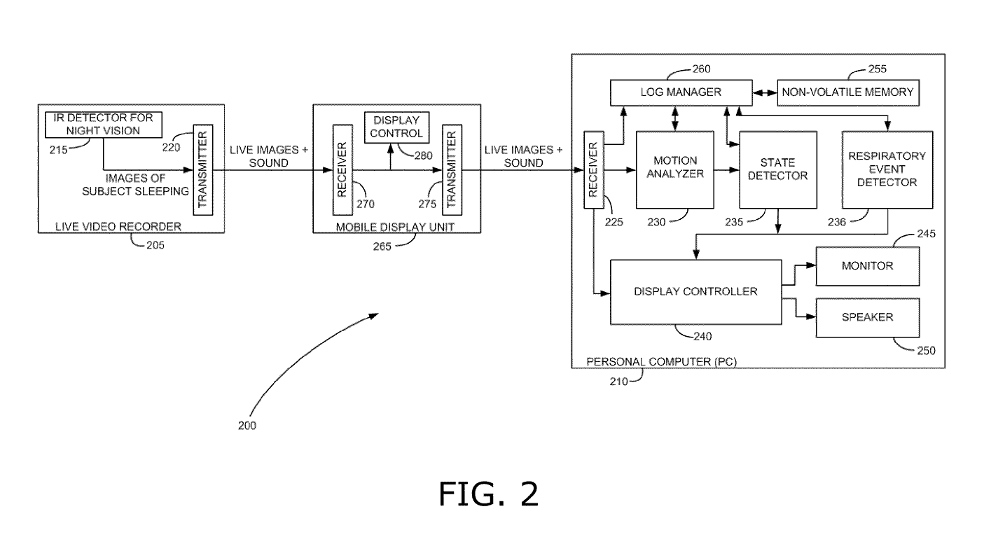

Reference is now made to FIG. 2, which is a simplified block diagram of a version of the monitoring system of FIG. 1 whereby the computing device is a personal (PC) computer that is connected to a mobile display unit, in accordance with a preferred embodiment of the present invention. Shown in FIG. 2 are the components of FIG. 1, together with mobile display unit 265 that includes a receiver 270 for receiving images and, optionally, sound from video recorder 205 over a wireless communication, and a transmitter 275 for transmitting the images and sound, if present, to personal computer 210. Personal computer 210 may be a personal computer, a laptop computer, a computer server or other processor-based system. Preferably, display unit 265 is connected to personal computer 210 via a USB interface or an IEEE 1394 connection, or such other standard digital transmission connection, which continuously communicates digitized display frame data from display unit 265 to personal computer 210. Optionally, display unit 265 may have its own display control 280 for viewing images directly on display unit 265.

Preferably, personal computer 210 runs a software application that processes the incoming images from display unit 265 and performs the operations of motion analyzer 230, state detector 235, and respiratory event detector 236. With this architecture, the present invention can be implemented using a video recorder and a separate display unit.

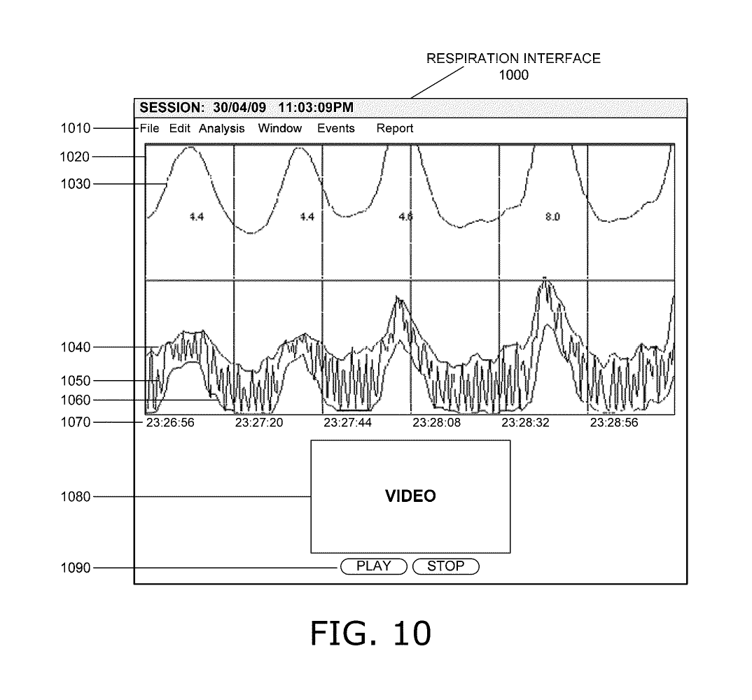

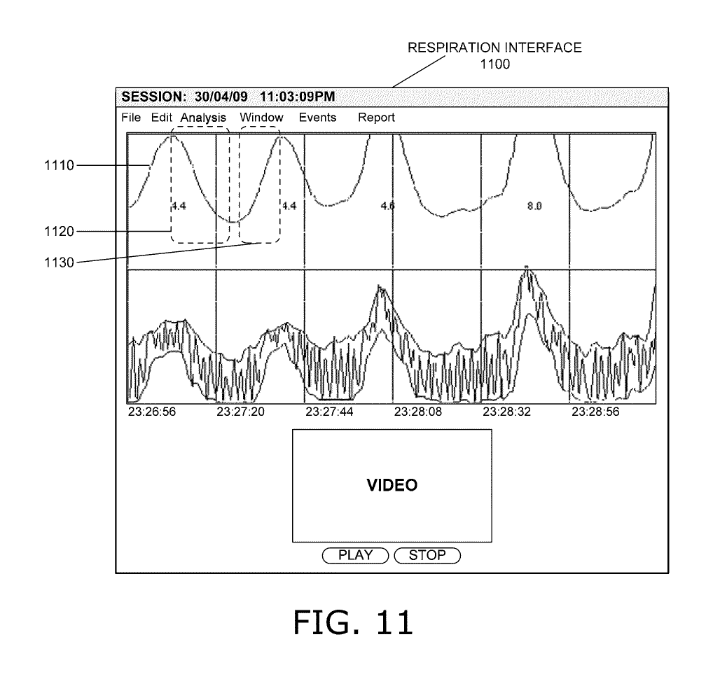

Another advantage of using a personal computer running special purpose software is the enhanced user interface that it provides, as illustrated in FIGS. 10 and 11. The PC offers the ability to design a detailed user interface that responds to full keyboard and mouse inputs.

Reference is now made to FIG. 3, which is a simplified block diagram of a real-time sleep monitoring system according to a second, embedded, architecture, wherein state and respiration analysis are performed by a video capture device, in accordance with a preferred embodiment of the present invention. Shown in FIG. 3 is an overall system including a live video recorder 305 and a mobile display device 310. Video recorder 305 captures live images of a subject sleeping.

Video recorder 305 is used to capture images of a subject sleeping. Preferably, video recorder has the ability to capture clear images within a dark surrounding, since this is typically the surrounding in which subjects sleep. To this end, video recorder 305 preferably includes an infrared (IR) detector 315, or such other heat sensitive or light sensitive detector.

Video recorder 305 includes a motion analyzer 330, which processes the recorded images to derive high-sensitivity motion detection. Results of motion analyzer 330 are passed to a state detector 335, which infers information about the state of the sleeping subject.

State information inferred by state detector 335 is passed to a transmitter 320 within video recorder 305, which transmits the state information to a receiver 325 within mobile display device 310. Preferably, transmitter 320 uses wireless communication so that video recorder need not be connected to mobile display device with physical wires.

Receiver 325 passes the received state information to a display controller 340, which controls a monitor 345. Display controller 340 activates monitor 345 to display state information, for viewing by a person remotely monitoring the sleeping subject.

Display controller 340 may continuously activate monitor 345, or may activate monitor 345 only when the state information is deemed to be significant. Display controller 340 may also activate mobile display device 310 to sound an alarm when the state information is deemed to be significant.

Video recorder 305 may optionally include non-volatile memory 355 and a log manager 360, which logs in memory 355 a time history of images, results of motion analysis and state data that describes the subject's sleep during the night. Such a time history can be used for post-analysis, to study the subject's sleep pattern and interference.

A respiratory event detector 336 reads motion analysis results stored in non-volatile memory 336 and further processes the motion analysis results data to detect respiratory events. Respiratory event detector 336 passes results data to transmitter 320 which transmits the state information to receiver 325 within mobile display device 310. Respiratory event detector 336 may also store results data in non-volatile memory 355 to be transmitted later.

The architecture in FIG. 3 performs the motion analysis, state detection, and respiratory event detection within video recorder 305. As such, mobile display device 310 can be a simple inexpensive display unit. It will be appreciated by those skilled in the art that whereas the system of FIG. 1 embeds the image processing within the display unit, the system of FIG. 3, in distinction, embeds the image processing within the video recorder.

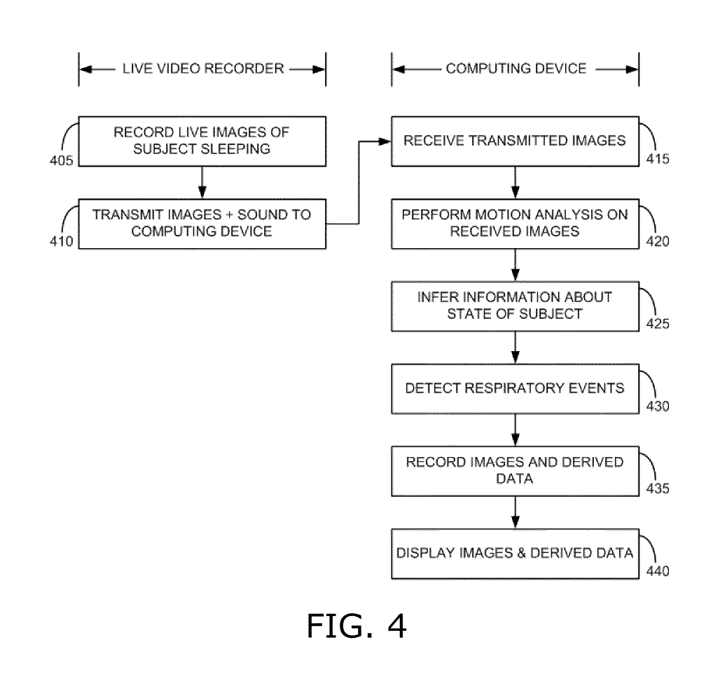

Reference is now made to FIG. 4, which is a simplified flowchart for a method of monitoring sleep by a computing device in real-time according to a first architecture, wherein state and respiration analysis are performed by the computing device, in accordance with a preferred embodiment of the present invention. The left column of FIG. 4 indicates steps performed by a live video recorder that records images of a subject sleeping, and the right column of FIG. 4 indicates steps performed by a computing device that monitors the sleeping subject.

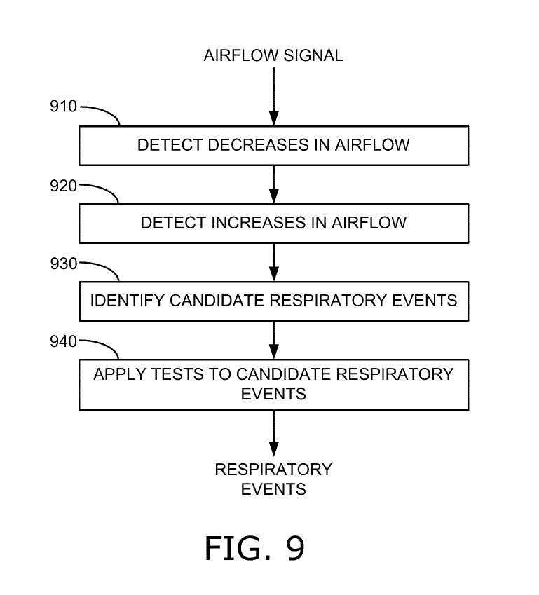

At step 405 the video recorder continuously records live images of the subject sleeping. Optionally, the video recorder may also continuously record sound. At step 410 the video recorder transmits the images, and optionally the sound, in real-time to the computing device, preferably via wireless communication. At step 415 the computing device receives the images. At step 420 the computing device performs motion analysis on the received images and derives high-sensitivity motion analysis data, as described in detail hereinbelow. At step 425 the computing device infers state information about the subject based on results of the motion analysis step. At step 430 respiratory events are detected. The processing performed at this step is described in further detail below with reference to FIGS. 8-9 below.

At step 435 some or all of the received images and derived data such as high-sensitivity motion analysis data, state data related to the subject's sleep and detected respiratory events are stored in a non-volatile memory such as non-volatile memory 155. Such stored data is preferably used for post-analysis, to derive statistics about the subject's sleep patterns and respiration, and to identify and evaluate other significant events that occurred while the subject slept.

At step 440 the computing device activates a monitor and displays images and derived data about the sleeping subject on a monitor. An example of a user interface used to display this data is provided in FIGS. 10-11 below. Optionally, at step 440 the computing device displays the received images on the monitor. Also, optionally, at step 440 the computing device activates a speaker and plays recorded sound on the speaker.

The architecture in FIG. 4 performs step 420 of motion analysis, step 425 of state inference, and step 430 of respiratory event detection on the computing device. As such, the computing device is preferably equipped with appropriate hardware to perform image processing.

Reference is now made to FIG. 5, which is a simplified flowchart for a method of monitoring sleep by a computing device in real-time according to a second architecture, wherein state and respiration analysis are performed by a video capture device, in accordance with a preferred embodiment of the present invention. The left column of FIG. 5 indicates steps performed by a live video recorder that captures images of a subject in bed, and the right column of FIG. 5 indicates steps performed by a display device that is used to monitor the subject.

At step 505 the video recorder captures live images of the subject sleeping. At step 510 the video recorder performs motion analysis of the captured images, preferably in real-time, and derives high-sensitivity motion analysis data. At step 515 the video recorder infers state information about the sleeping subject, based on the results of motion analysis step 510. At step 516 the video recorder accesses the stored motion analysis data and further analyzes the data to detect respiratory events. At step 520 the video recorder transmits the received images, inferred state information and/or respiration data to the mobile display device, preferably via wireless communication. In one embodiment, received images, state information and respiration data are transmitted. In another embodiment, a selection of data is transmitted, typically in response to user commands to display one or the other types of information.

Optionally, at step 525, the video recorder stores or logs the recorded images and derived data including inferred state and respiration data relating to the subject's sleep in a non-volatile memory such as non-volatile memory 355. \Such information is preferably used for post-analysis diagnosis, to study the subject's sleep patterns and disturbances.

At step 530 the display device receives the images, and derived data transmitted at step 520. At step 535 the display device activates a monitor and displays the received images and/or derived data on the monitor.

The architecture in FIG. 5 performs the motion analysis step 510, the state inference step 515, and the respiration event detection step 516 at the video recorder, and not at the mobile display device. As such, the mobile display device can be a simple and inexpensive display unit.

### Motion Detection

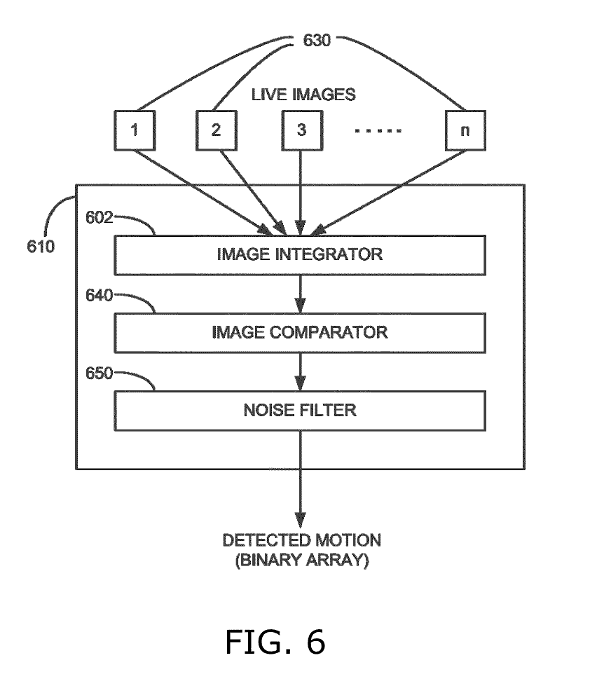

The operation of motion analyzers 130, 230 and 330 in FIGS. 1-3, respectively, and motion analysis steps 420 and 510 in FIGS. 4 and 5, respectively, will now be described in detail. Reference is now made to FIG. 6, which is a simplified block diagram of a high-sensitivity motion analyzer 610, in accordance with a preferred embodiment of the present invention. Motion analyzer 610 continuously receives as input a plurality of images, I₁, I₂, . . . , Iₙ, and produces as output a binary array, B(i, j), of one's and zero's where one's indicate pixel locations (i, j) at which motion has been detected.

As shown in FIG. 6, motion analyzer 610 includes three phases; namely, (i) an image integrator 620 that integrates a number, n, of live images 630 recorded by a video recorder, (ii) a frame comparator that compares pixel values between images, and (iii) a noise filter that removes noise captured in the video recorder. Operating conditions of motion analyzer 610 are such that the level of noise may be higher than the level of movement to be detected, especially in low light surroundings. Since motion analyzer 610 is required to detect subtle movement, a challenge of the system is to appropriately filter the noise so as to maximize motion detection intelligence.

Typically, pixel values are specified by a rectangular array of integer or floating point data for one or more color channels. Familiar color systems include RGB red-green-blue color channels, CMYK cyan-magenta-yellow-black color channels and YUV luminance-chrominance color channels. For the present analysis, noise for color channel data is modeled as being Gaussian additive; i.e., if I(i, j) denotes the true color data at pixel location (i, j) for a color channel, and if G(i, j) denotes the color value measured by a video recorder, then

G(i,j)=I(i,j)+ε(i,j), where ε(i,j)˜N(μ,σ²), (1)

with mean μ, which is assumed to be zero, μ=0, and variance σ². Preferably, the values I(i, j) are luminance values.

Image integrator 620 receives as input a time series of n images, with pixel data denoted G₁(i, j), G₂(i, j), . . . , Gₙ(i, j), and produces as output an integrated image I(i, j). Preferably, image integrator 620 reduces the noise level indicated in Equation (1) by averaging. Thus if I(i, j) denotes the color data at pixel location (i, j) after integrating the n images, then the noise level can be reduced by defining:

$$I ( i , j ) = \frac{1}{n} {\sum }_{k = 1}^{n} {G}_{k} ( i , j ) . \qquad ( 2 )$$

As each additional image Gₙ₊₁(i, j) is integrated within image integrator 620, the averaged pixel values are accordingly incremented dynamically as follows:

$$I ( i , j ) ← I ( i , j ) + \frac{{G}_{n + 1} ( i , j ) - {G}_{1} ( i , j )}{n} . \qquad ( 3 )$$

For the present invention, an approximation to Equation (3) is used instead; namely,

$$I ( i , j ) ← I ( i , j ) + \frac{{G}_{n + 1} ( i , j ) - I ( i , j )}{n} , \qquad ( 4 )$$

where I(i, j) has been used instead of G₁(i, j). The advantage of Equation (4) over Equation (3) is that use of Equation (4) does not require maintaining storage of the raw image data G₁(i, j), G₂(i, j), . . . , Gₙ(i, j) over a history of n images.

An advantage of averaging image data, as in Equation (2) above, is the elimination of noise. However, a disadvantage of averaging is that it tends to eliminate subtle movements, and especially periodic movement, making it hard to derive estimates of motion by comparing two images close in time. Thus in order to compensate for averaging, the present invention compares two images that are separated in time by approximately 1 second. In turn, this requires that a circular storage buffer of integrated images I(i, j) is maintained over a corresponding time span of approximately 1 second. For a video recording frame rate of, say, 15 frames per second, this corresponds to a circular buffer of approximately 15 images.

Image comparator 640 receives as input the integrated images I(i, j) generated by image integrator 620, and produces as output a rectangular array, Δ(i, j), of binary values (one's and zero's) that correspond to pixel color value differences. Image comparator 640 determines which portions of the images are moving, and operates by comparing two integrated images that are approximately 1 second apart in time. Preferably, image comparator 640 uses differential changes instead of absolute changes, in order to avoid false movement detection when global lighting conditions change.

Denote by IA(i, j) and IB(i, j) two integrated images that are approximately one second apart in time, and that are being compared in order to extract motion information. Absolute differences such as |IA(i,j)−IB(i,j) are generally biased in the presence of a change in global lighting conditions. To avoid such a bias, image comparator 640 preferably uses differential changes of the form:

Δ(i,j)=|IA(i,j)−IA(i−δ₁,j−δ₂)|−|IB(i,j)−IB(i−δ₁,j−δ₂)|. (5)

Equation (5) incorporates both a spatial difference in a gradient direction (δ₁, δ₂), and a temporal difference over an approximate 1 second time frame. It is noted that a spatial difference generally eliminates global biases. Preferably, image comparator 640 uses a sum of several such terms (5) over several different gradient directions.

After computing the differences Δ(i, j) at each pixel location (i,j), image comparator 640 preferably uses a threshold value to replace Δ(i, j) with 1 for values of δ greater than or equal to the threshold value, and to replace Δ(i, j) with 0 for value of δ less than the threshold value. As such the output of image comparator is a binary array, B(i, j), with values B=0 or B=1 at each pixel location (i, j).

The output of image comparator 640 is passed to noise filter 650 for applying active noise filters. Noise filter 650 receives as input the binary array representing pixel color value differences produced by image comparator 640, and produces as output a corresponding noise-filtered binary array. Operation of noise filter 650 is based on the premises that (i) motion generally shows up in multiple consecutive image differences, and not just in a single image difference; and (ii) motion generally shows up in a cluster of pixels, and not just in a single isolated pixel. Accordingly, noise filter 650 modifies the binary array B(i,j) by zeroing out values B(i, j)=1 unless those values of one's have persisted throughout some number, m, of consecutive comparison arrays B over time; and (ii) erosion is applied to the thus modified array B(i, j) so as to zero out values of B(i,j)=1 at isolated pixels locations (i, j).

The binary array B(i, j) output by noise filter 650 identifies motion within the image; i.e., the pixel locations where B(i, j)=1 correspond to locations where motion is detected.

### State Detection

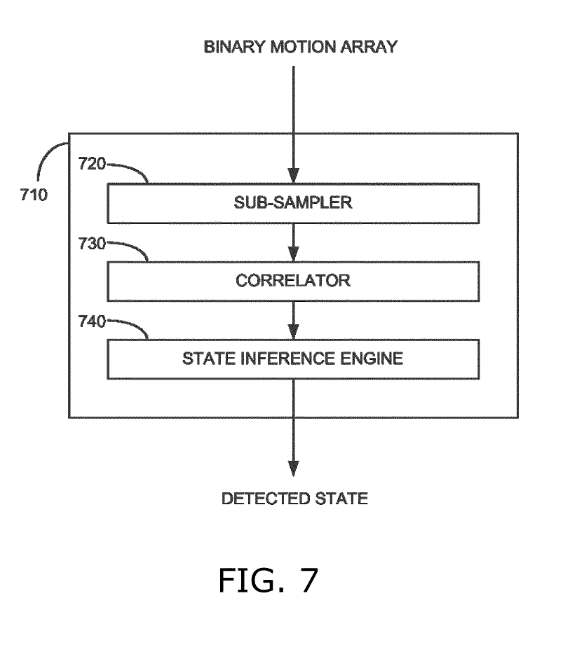

The operation of state detectors 135, 235 and 335 in FIGS. 1-3, respectively, and state detection steps 425 and 515 in FIGS. 4 and 5, respectively, will now be described in detail. Reference is now made to FIG. 7, which is a simplified block diagram of a state detector, in accordance with a preferred embodiment of the present invention. Shown FIG. 7 is a state detector 710 that receives as input a binary array, B(i, j), of one's and zero's indicating pixel locations where motion is detected. Such an array is normally output from motion detector 610. State detector 710 produces as output one or more automatically inferred states, that describe the subject being monitored.

State detector 710 performs three phases, as follows: (i) a sub-sampler 720 sub-samples the binary motion array to reduce it to a smaller resolution, (ii) a correlator 730 derives a measure of correlation between the current sub-sampled binary motion array and previous such arrays corresponding to times between 2 and 6 seconds prior to the current time, and (iii) a state inference engine 740 uses the measure of correlation to infer state information about the subject being monitored.

Based on the motion detection arrays B(i, j) output by the motion detection phase, pattern analysis is performed to detect if the motion exhibits repetitive patterns. Generally, a repetitive pattern indicates that the subject is sleeping, a non-repetitive pattern indicates that the subject is awake, and no motion for a period of 20 seconds indicates a state of alert.

Sub-sampler 720 accepts as input a binary array, B(i, j) of one's and zero's, and produces as output a binary array, BS(x, y), of smaller dimensions, that corresponds to a sub-sampled version of the input array, B(i, j). In accordance with a preferred embodiment of the present invention, sub-sampler 720 proceeds by sub-sampling the binary motion detection arrays B(i, j) to reduced resolution arrays, BS(x, y), of dimensions K×L pixels, wherein each sub-sampled pixel location (x, y) within BS corresponds to a rectangle R(x, y) of pixel locations (i, j) in a local neighborhood of the pixel location corresponding to (x, y) within B. Specifically, the sub-sampling operates by thresholding the numbers of pixel locations having B(i, j)=1 within each rectangle, so that BS(x, y) is assigned a value of 1 when the number of pixel locations (i, j) in rectangle R(x, y) satisfying B(i, j)=1 exceeds a threshold number.

Preferably, the sub-sampled binary arrays BS are stored in a circular queue that spans a timeframe of approximately 6 seconds.

Correlator 730 accepts as input the sub-sampled arrays BS(x, y) produced by sub-sampler 720, and produces as output measures of correlation, C, ranging between zero and one. Correlator 730 preferably derives a measure of correlation, C, at each time, T, as follows:

$$C = max \{ \frac{M ( t )}{M ( t ) + N ( t )} ∷ T - 6 \leq t \leq T - 2 \} , where \qquad ( 6 )$$

M(t) is the number of sub-sampled pixel locations (x, y) at which BS(x,y)=1 at the current time and BS(x, y)=1 at time t (a match), and N(t) is the number of sub-sampled pixel locations (x, y) at which B(x,y)=1 at the current time and BS(x, y)=0 at time t (a non-match). The restriction of t to being at least 2 seconds away from T is to ignore the high correlation between any two images that are recorded at almost the same time. It will be appreciated by those skilled in the art that the value of M(t) and N(t) can be efficiently computed by using conventional AND and NOT Boolean operations.

State detection engine 740 accepts as input the measures of correlation generated by correlator 730, and produces as output one or more inferred states. Based on the time series of the correlation measures, C, state detection engine 740 proceeds based on empirical rules.

As mentioned hereinabove, in accordance with a preferred embodiment of the present invention, repetitiveness is used to characterize a subject's sleep. If the subject is moving, which is manifested in a lack of repetitiveness, then he is more likely to be in a soft sleep; whereas if the subject does not move for a specific amount of time, which is manifested in a presence of repetitiveness, then he is more likely to be in a deep sleep. The correlation measures, C, are used as indicators of repetitive motion.

An example set of empirical rules that governs state determination is based on the premise that if C exceeds a threshold value, then the motion is repetitive, and was repeated at least 2 seconds before the current time. If C remains large for more than 60 seconds, then the person is sleeping. Otherwise, the person is awake. If no movement is detected for 20 seconds or longer, a state of alert is identified and preferably an alarm is sounded.

### Respiratory Event Detection

The operation of respiratory event detectors 136, 236, 336, and 1291 in FIGS. 1-3, and 12 respectively, and respiratory event detection steps 455, 516, and 1350 in FIGS. 4, 5, and 13 respectively, will now be described in detail. Reference is now made to FIG. 8, which is a simplified block diagram of a respiratory event detector 810, in accordance with a preferred embodiment of the present invention. Respiratory event detector 810 receives as input the binary array, B(i, j), of one's and zero's produced as output by high sensitivity motion analyzer 610 and produces as output a movement signal, an airflow signal and a set of zero or more detected respiratory events for a period of time being analyzed.

As previously discussed, the result of motion analysis steps 420 and 510 in FIGS. 4 and 5 respectively is stored in binary array B(i, j). In one embodiment, the resolution of the array B is equal to the resolution of the received image. Thus, each pixel in a received image corresponds to one position (i, j) in B(i, j). The binary value of each position, or pixel, (i, j), in B(i, j) indicates if the system has detected movement in that pixel. If movement has been detected the value of position (i,j) in B(i, j) is one (1). If no movement is detected in that position the value is set to zero (0).

A movement signal generator 820 uses stored arrays, B(i, j), to generate a movement signal, M(t), where M(t) is a function that generates a single value for each received image that represents the total motion in the image. First, signal generator 820 calculates a raw movement signal, RM(t), by determining the number of nonzero pixels in B(i, j). In other words, RM(t) is the number of pixels in the array B(i, j) where the system has detected movement. B(i, j) in this case refers to the motion array for time t.

In a preferred embodiment, movement signal generator 820 then applies a smoothing filter, to further reduce noise, to RM(t) to produce movement signal M(t). In a preferred embodiment, the smoothing filter averages n adjacent values of M(t). The value of n is based on the sample rate, i.e. the number of images received per second. For example, in one embodiment, ten samples received during a one second interval are averaged. Thus, M(t) is obtained by:

$$M ( t ) = \frac{1}{n} {\sum }_{k = 1}^{n} RM ( t - rnd ( n / 2 ) + k ) . \qquad ( 7 )$$

For each t, this equation yields a value for M(t) that is an average across the n adjacent values of RM(t) centered at t. The function rnd(n/2) rounds the value n/2 up to the nearest integer in the case that n is an odd integer.

Next, an envelope detector 830 calculates a movement envelope for the movement signal, M(t). Envelope detector 830 receives as input the movement signal M(t) and produces as output two signals, or series, an upper envelope Mᵤₚₚₑᵣ(t) and a lower envelope, M_{lower}(t) as follows:

Mᵤₚₚₑᵣ(t) is the maximum value that M(t) takes on during a time interval, I, centered at time t.

M_{lower}(t) is the minimum value that M(t) takes on during a time interval, I, centered at time t.

A preferred value for time interval I is 0.5 seconds, but longer or shorter values can be used. Thus, the upper and lower movement envelopes are line segments that, taken together, enclose the values of M(t).

An airflow signal generator 840 creates an airflow signal, S(t), by adding the lower and upper value of the movement envelope at each point. Thus:

$$S ( t ) = {M}_{upper} ( t ) + {M}_{lower} ( t ) \qquad ( 8 )$$

Air flow signal, S(t), represents the amount of air flowing into the lungs of the subject over time. When the subject breathes deeply the corresponding movement and hence the values of the airflow signal increase during the period of deep breathing; when the subject breathes shallowly, or not at all, his/her movement decreases and hence the values of the airflow signal decrease during the corresponding period.

A respiratory event classifier 850 receives airflow signal S(t) and analyzes it to detect respiratory events. The operation of respiratory event classifier 850 is described hereinbelow with reference to FIG. 9.

Reference is now made to FIG. 9, which is a simplified flow diagram of a method for detecting respiratory events. Respiratory events, as identified by respiratory event classifier 850, occur when a significant decrease in the airflow signal is followed by a significant increase in the airflow signal. The first step 910 is to detect significant decreases in the air flow signal. This is performed, by identifying the intervals [t0, t1] where the derivative of the airflow signal, S(t) is uniformly negative, in other words interval [t0, t1] satisfies:

∀t,t0<t<t1,(S(t)−S(t+δ))<0 (9)

Here δ is the sample interval; in other words, if S(t) is the value of airflow signal S(t) at time t, then S(t+(t+δ) is the value of the airflow signal that corresponds to the next received image.

At step 920 significant increases in the air flow signal are detected. This is performed, by identifying the intervals [t0, t1] where the derivative of the airflow signal is uniformly positive, in other words interval [t0, t1] satisfies:

∀t,t0<t<t1,(S(t)−S(t+δ))>0 (10)

Here again δ is the sample interval such that if S(t) is the value of airflow signal S(t) at time t, then S(t+δ) is the value of the airflow signal corresponding to the next received image.

At step 930 candidate respiratory events are identified. Each case where a significant decrease in the airflow signal, as detected at step 910, is followed by a significant increase in the airflow signal, as detected at step 920 is considered a candidate respiratory event. A candidate event then is a time interval that begins when the derivative of an airflow signal becomes negative until the derivative of the airflow signal reverses and becomes positive.

However, not all the candidate respiratory events qualify as true respiratory events. From a clinical point of view, a decrease in the airflow is not considered a respiratory event if it does not have a significant impact on the saturation of oxygen in the blood. Thus, at step 940 empirically derived tests are applied to the candidate events in order to discard events that are determined to be insignificant. The remaining candidate events are then deemed to be true respiratory events. In one embodiment, the empirical tests are: duration of the decrease in the airflow signal and the derivative of the airflow signal. Additional or different tests may also be applied to the candidate respiratory events, either to discard false events or to identify true respiratory events, within the scope and spirit of the subject invention.

Duration of the decrease. If the interval [t0, t1] when a decrease in the air flow signal occurred, as detected at step 910, lasts less than a threshold period of time, referred to as the Decrease Duration Threshold, the interval will be discarded because the corresponding decrease in the air flow is too brief to significantly influence the saturation of the oxygen in the blood.

Derivative of the airflow signal. The average derivative of the airflow signal, S(t), during a candidate interval [t0, t1] must initially, during the period of decreasing respiration, exceed a negative threshold, referred to as the Negative Derivative Threshold. In the next phase, when respiration increases, the average derivative must exceed a Positive Derivative Threshold. The derivative threshold test is calculated using an approximation of the average derivative of S(t) over the interval [t0, t1] as follows:

During the initial period of decreasing airflow:

$$\frac{max [ {S}^{′} ( t ) ] - min [ {S}^{′} ( t ) ]}{dur [ t 0 , t 1 ]} \leq Negative Derivative Threshold \qquad ( 11 )$$

And, during the following period of increasing airflow:

$$\frac{max [ {S}^{′} ( t ) ] - min [ {S}^{′} ( t ) ]}{dur [ t 0 , t 1 ]} \geq Positive Derivative Threshold \qquad ( 12 )$$

where

max[S′(t)] is the maximum value of the derivative of S(t) over the interval [t0, t1],

min[S′(t)] is the minimum value of the derivative of S(t) over the interval [t0, t1], and

dur[t0, t1] is the duration of the time interval [t0, t1], typically measured in seconds.

In one embodiment, the values of the two thresholds used in step 940, Decrease Duration Threshold and Derivative Threshold, are established using clinical test results obtained using polysomnography. Essentially, the events detected using the subject invention are compared with those obtained using a polysomnography apparatus in a clinical environment such as a sleep center or a hospital. The, using the subject invention to perform a comparable analysis the various thresholds are systematically varied to yield the best comparative results. This is performed over a number of sleep sessions involving different subjects to further tune the threshold values.

In a preferred embodiment, the Decrease Duration Threshold is set preferably to ten seconds. Moreover, in a preferred embodiment, the negative derivative threshold is set to −0.5 and the positive derivative threshold is set to 0.7.

### User Interface

Reference is now made to FIG. 10, which is an illustration of a user interface, referred to as respiration interface 1000, for monitoring and analyzing respiration and respiratory events, in accordance with a preferred embodiment of the present invention. Respiration interface 1000 is displayed on the video monitor of a computing device, for a system of the present invention designed according to the architecture of FIGS. 1-2 and 12. The computing device runs a special software application that performs steps 415-440 of FIG. 4 or steps 1340-1375 of FIG. 13.

Respiration interface 1000 includes a menu bar 1010 that includes a selection of menu items. Functions that can be activated via the menu items include selecting a sleep session to review, setting a start time of a time interval within a sleep session to review, setting an end time of a time interval within a sleep session to review, selecting a duration to review, setting display options including whether to display candidate events, detected events, obstructive apneas, central apneas, natural movements, suspicious events, whether to play recorded sound, and so forth. It may be appreciated by one skilled in the art that user interface techniques other than a menu bar can equally be used to enable a user to activate such functions, including inter alia visible entry fields, a vertical menu or a floating control window.

Window 1020 displays derived respiration data including an airflow signal 1030, as computed using Equation 8, a movement signal 1050, M(t), as computed using equation 7, an upper envelope 1040, Mᵤₚₚₑᵣ(t), of the movement signal, M(t), a lower envelope 1060, M_{lower}(t) of the movement signal, M(t) and time values 1070 for the recorded session running along the horizontal axis.

A video window 1080 enables the user to view the video corresponding to the respiration data. The user uses a set of video controls 1090 to control the video. In example respiration interface 1000 video controls 1090 are Play and Stop, however additional controls such as Pause, Resume, Fast Forward, Rewind can additionally be offered. where such controls including Play and Stop.

Reference is now made to FIG. 11, which is an illustration of a user interface, referred to as respiration interface 1100, for monitoring and analyzing respiration and respiratory events, in accordance with a preferred embodiment of the present invention. Respiration interface 1100 is a version of respiration interface 1100 in which an option to display detected respiration events has been activated. A rounded rectangular bounding box 1120 encloses an area of a movement signal 1110 where there is a significant decrease of airflow signal 1110, as detected in step 910 of FIG. 9. A second rectangular bounding box 1130 encloses an area of a movement signal 1110 where there is a corresponding significant increase of airflow signal 1110, as detected in step 920 of FIG. 9.

### Additional Architecture

A third architecture that relies on two computing devices to analyze the stream of images of a sleeping subject is presented hereinbelow with reference to FIGS. 12-13. In this processing, the image processing performed on a stream of images captured by a video recorder is allocated between the two computing devices. In a preferred embodiment, the first computing device is typically a personal computer, laptop computer or tablet computer that is used in a home. The first computing device receives images from a video recorder, performs motion analysis processing on the captured images and then transmits the motion analysis results data and, optionally, the captured images across a network to a second computing device for further processing. The second computing device is typically a high performance computer server but may be any suitably equipped computing device. The second computing device performs state inference and respiratory event detection. The derived data, such as inferred states and detected respiratory events, may be summarized and formatted into reports which are provided across a network to a suitably equipped individual of service such as the subject, a doctor, technician, hospital worker or sleep clinic.

Reference is now made to FIG. 12, which is a simplified block diagram of a real-time sleep monitoring system 1200 according to a third, wherein motion analysis is performed by a first computing device and state detection and respiratory event detection are performed by a second computing device, in accordance with a preferred embodiment of the present invention. Included in system 1200 is a live video recorder 1205, which records images of a subject while the subject is sleeping, a first computing device 1210, which receives the recorded images and performs high-sensitivity motion analysis on the recorded images, a network 1250 across which first computing device 1210 communicates with a second computing device 1270. Second computing device 1270 receives motion analysis results data, and potentially images from first computing device 1210 and infers information about the state of the subject and performs respiratory event detection. Examples of suitable computing devices 1210 and 1270 include inter alia a personal computer, a tablet computer, a computer server, a laptop computer, smart phone or other processor-based system. In addition, second computing device 1270 may be implemented as a network-based service performed by one or more computers that connects to first computing device 1210 across network 1250.

Video recorder 1205 is similar to video recorder 105 and preferably includes an infrared (IR) detector 1215 or such other heat sensitive or light sensitive component used to enhance night vision and a transmitter 1220, which transmits the recorded images, preferably in real-time, to first computing device 1210. Transmitter 1220 transmits the recorded images using wireless or wired communications links.

A receiver 1225 in second computing device 1270 receives the transmitted images. The images are passed to a motion analyzer 1230, which performs high sensitivity motion detection as described in detail hereinabove. The results of motion analyzer 1230 are passed to a network interface 1265 that communicates the results of the motion analysis across a network 1250 to a network interface in second computing device 1270. Images received from receiver 1225 and derived data from motion analyzer 1230 are, optionally, also stored in a non-volatile memory 1255 which is managed by a log manager 1260 and are, optionally, passed to a display controller 1240 for display on a monitor 1245.

Network 1250 may be any wide area network, including the Internet, or local area network, including wireless local area networks. Network 1250 also includes communications between devices using physical media such as USB drives, DVD or CD ROM or CD RW. Essentially, network 1250 includes any media or mechanism capable of enabling digital data exchange between first computing device 1210 and second computing device 1270 and between second computing device 1270 and third party computers and services.

Network interface 1275 receives the motion analysis data across network 1250 and provides it to a state detector 1290, which infers a state of the subject, as described in detail hereinabove. Detected states may include inter alia “sleeping”, “awake”, “light sleep”, and “deep sleep”.

State information inferred by state detector 1290 is stored in a non-volatile memory 1285 which is managed by a log manager 1280 and is, optionally, passed to a display controller 1295 for display on a monitor 1296.

A respiratory event detector 1291 further processes motion information stored in non-volatile memory 1285 to detect respiratory events. The further processed motion information and detected respiratory events are stored in non-volatile memory 1285 and may be, optionally, displayed on monitor 1296 via display controller 1295. The operation of respiratory event detector 1291 is described in further detail above with reference to FIGS. 8-9.

A report generator 1292 processes derived data and images stored in non-volatile memory 1285 to produce summary reports. Such reports may be provided using network interface 1275 to a computing device at the subject's location or to a computing device at a third party location across network 1250.

Log managers 1260 and 1280 store and retrieve in non-volatile memories 1255 and 1285, respectively, a time history of images, and derived information such as state information, motion information, detected respiratory events, and other information that describes the subject's sleep. Such information can be used for post-analysis, including statistical analysis of sleep patterns and interference. Log managers 1260 and 1280 may also store and retrieve reports and intermediate information such as partially processed data. Log managers 1260 and 1280 may be implemented as database managers or other software modules or devices capable of storing structured data onto physical media.

Non-volatile memories 1255 and 1285 may include virtually any mechanism usable for storing and managing data, including but not limited to a file, a folder, a document, one or more database file(s), and the like that are stored on physical media such as disk drives, USB drives, DVD RW, and CD RW. Non-volatile memories 1255 and 1285 may further represent a plurality of different data stores. For example non-volatile memories 1255 and 1285 may be implemented as an image or video database that stores captured digital images and/or digital videos, a motion information database, a state information database, and a respiratory event database. Further, non-volatile memories 1255 and 1285 may also include network storage or cloud storage in which the physical storage media is accessed across a network.

A key objective of this third architecture, represented in system 1200, is to provide summary information about the subject's sleep, which may include images and derived data across network 1250 to third parties via suitable networked computers. Such third parties include doctors, hospital workers, and sleep specialists and technicians. To provide the derived data, network interface 1275 may be capable of communicating using a variety of network protocols and interfaces, including inter alia file transfer protocol (FTP), email attachments, web pages and through client-server applications that respond to request for data from a doctor or other third party.

Additionally, derived data can be communicated back from second computing device 1270 to first computing device 1210 to display to the subject or to store in non-volatile memory 1255 for later use.

Reference is now made to FIG. 13, which is a simplified flowchart for a method of monitoring sleep according to a third architecture, wherein motion analysis is performed by a first computing device, and state detection and respiratory event detection are performed by a second computing device, in accordance with a preferred embodiment of the present invention. The left column of FIG. 13 indicates steps performed by a live video recorder, such as video recorder 1205, that records images of a subject sleeping; the middle column indicates steps performed by first computing device, such as first computing device 1210; and the right column of FIG. 12 indicates steps performed by a second computing device, such as second computing device 1270.

At step 1305 the video recorder continuously records live images of the subject sleeping. Optionally, the video recorder may also continuously record sound. At step 1310 the video recorder transmits the images, and optionally the sound, in real-time to the first computing device. At step 1315 the first computing device receives the images, and sound, if transmitted. At step 1320 the first computing device analyzes the received images and derives high-sensitivity motion analysis data, as described in detail hereinabove. At step 1325 the first computing device, optionally, records the derived motion analysis data, and, also optionally, the received images, to a non-volatile memory. At step 1330 the first computing device transmits across a network the derived motion analysis data and, optionally, the received images to a second computing for further processing. At step 1330, the first computing device may also receive across a network from the second computing device additional derived data regarding the subject's sleep. Such additional derived data typically includes inferred state and respiratory event information. At step 1335, the received images may be displayed. Also at step 1335, derived data may be displayed. In one embodiment, the subject or user of the first computing device controls whether or not to display the received images and derived data.

At step 1340 the second computing device receives the motion analysis results data, and, optionally, the received images from the first computing device. At step 1345 the second computing device infers state information about the subject based on the received motion analysis results data received in the preceding step. At step 1350 respiratory events are detected. The processing performed at this step is described in further detail with reference to FIGS. 8-9 above.

At step 1355 the derived data including high-sensitivity motion analysis data, state data related to the subject's sleep and detected respiratory events, and any received images of the subject sleeping are stored in a non-volatile memory such as non-volatile memory 1285. Such stored data is preferably used for post-analysis to produce summary information about the subject's sleep.

At step 1360, optionally, some or all of the derived data is transmitted across the network to the first computing device for purposes of display.

At step 1365 a report generator such as report generator 1292 generates, or produces, reports about the subject's sleep. The summary information may be in the form of screen displays such as the example in FIGS. 10-11, reports, statistics, or data files. The summary information typically includes state data related to the subject's sleep, detected respiratory events and may include motion data and selected images or sequences of images depicting the subject sleeping.

At step 1370, the second computing devices provides the reports generated in the preceding step across the network. As previously discussed, typically reports are provided to the subject via the first computing device or to doctors or diagnosticians using network-connected computers in their offices or homes across a network such as network 1250.

At step 1375 the second computing device, optionally, displays the derived data and summary information on a monitor. An example of a user interface used to display this data is provided in FIGS. 10-11 above.

It may be appreciated by one skilled in the art, that the image processing functions, namely the motion analysis performed at step 1320, the inferring of sleep states performed at step 1345 and the detection of respiratory events performed at step 1350, may be allocated differently among the two computing devices, subject to the constraint that motion analysis is performed first, without departing from the scope and spirit of the present invention. For example, in one embodiment motion analysis may be performed in the second computing device.

### Additional Considerations

The ability of the present invention to automatically detect respiration events such as apneas leads naturally to a variety of auxiliary sleep-related functions that the present invention enables. In general, it will be appreciated that knowledge of the respiration of a subject being monitored in bed enables a system to perform services that are adapted to the subject's current respiration.

One such service is to signal an alarm if the subject is experiencing a respiratory event such as an apnea. Another such service is that a summary of a night's sleep, i.e. a session, can be recorded or printed and provided to a doctor or other sleep analyst or technician for review. Such data can assist further diagnosis and treatment.

In reading the above description, persons skilled in the art will realize that there are many apparent variations that can be applied to the methods and systems described. Thus it may be appreciated that although FIGS. 1-2, and 12 allow the use of wireless communication between the video recorder and the computing device, other modes of communication may be used instead. For example, IP cameras that use digital networks, which may or may not be wireless, can be used for image capture.

In the foregoing specification, the invention has been described with reference to specific exemplary embodiments thereof. It will, however, be evident that various modifications and changes may be made to the specific exemplary embodiments without departing from the broader spirit and scope of the invention as set forth in the appended claims. Accordingly, the specification and drawings are to be regarded in an illustrative rather than a restrictive sense.

What is claimed is:

1. Apparatus for automatically monitoring sleep, comprising:
a video recorder for recording live images of a subject sleeping, comprising a transmitter for transmitting the recorded images to a computing device; and
a computing device communicating with said video recorder transmitter, comprising:
a receiver for receiving the transmitted images;
a motion analyzer for performing motion analysis of the subject based on the received images;
a respiratory event detector for (1) computing an air flow signal that represents the amount of air flowing into the lungs of the subject over time from the results of said motion analysis, and (2) automatically detecting respiratory events based on said airflow signal, wherein a respiratory event is a breathing disturbance; and
a monitor for displaying the respiratory events experienced by the subject, detected by said computing device.

2. The apparatus of claim 1 wherein said monitor also displays the received images.

3. The apparatus of claim 1 wherein said respiratory event detector identifies candidate respiratory events, wherein a candidate respiratory event is indicated by the derivative of the airflow signal being uniformly negative during a first interval followed by a second interval during which the derivative of the airflow signal is uniformly positive.

4. The apparatus of claim 3 wherein a respiratory event is determined from a candidate respiratory event by applying a threshold to the first interval, the period of time during which the airflow signal is uniformly negative

5. The apparatus of claim 3 wherein a respiratory event is determined from a candidate respiratory event by applying a threshold to the average derivative of the airflow signal during said first time interval or to the average derivative of the airflow signal during the second time interval.

6. The apparatus of claim 3 wherein said monitor also displays derived respiratory information, wherein said derived respiratory information is a member of the group consisting of respiratory events, candidate respiratory events, and the air flow signal.

7. The apparatus of claim 1 wherein said computing device further comprises:
a non-volatile memory; and
a log manager for selectively storing the received images and derived respiratory information in the non-volatile memory for subsequent post-analysis or display, wherein said derived respiratory information is a member of the group consisting of the airflow signal and the detected respiratory events.

8. A computer implemented method for automated sleep monitoring, comprising:
recording live images of a subject sleeping;
transmitting the recorded images to a computing device;
receiving the transmitted images at the computing device;
performing motion analysis of the subject based on the received images;
computing an air flow signal that represents the amount of air flowing into the lungs of the subject over time from the results of said motion analysis;
automatically detecting respiratory events based on said airflow signal, wherein a respiratory event is a breathing disturbance; and
displaying the respiratory events experienced by the subject on a monitor.

9. The method of claim 8 wherein said displaying also displays the received images.

10. The method of claim 8 wherein said detecting identifies candidate respiratory events, wherein a candidate respiratory event is indicated by the derivative of the airflow signal being uniformly negative during a first interval followed by a second interval during which the derivative of the airflow signal is uniformly positive.

11. The method of claim 10 wherein a respiratory event is determined in part from a candidate respiratory event by applying a threshold to the first interval, the period of time during which the airflow signal is uniformly negative

12. The method of claim 10 wherein a respiratory event is determined from a candidate respiratory event by applying a threshold to the average derivative of the airflow signal during said first time interval or to the average derivative of the airflow signal during the second time interval.

13. The method of claim 10 wherein said displaying also displays derived respiratory information wherein said derived respiratory information is a member of the group consisting of respiratory events, candidate respiratory events, and the air flow signal.

14. The method of claim 8 further comprising selectively storing the received images and derived respiratory information in a non-volatile memory for subsequent post-analysis or display, wherein said derived respiratory information is a member of the group consisting of the airflow signal and the detected respiratory events.

15. A system for automatically monitoring sleep, comprising:
a video recorder for recording live images of a subject sleeping, comprising a transmitter for transmitting the recorded images to a computing device; and
a first computing device communicating with said video recorder transmitter, comprising:
a receiver for receiving the transmitted images;
a motion analyzer for performing motion analysis of the subject based on the received images; and
a network interface for transmitting the results of said motion analysis across a network to a second computing device; and
a second computing device communicatively coupled with said first computing device, comprising:
a second network interface for receiving the results of said motion analysis across the network from the first computing device;
a respiratory event detector for (1) computing an air flow signal that represents the amount of air flowing into the lungs of the subject over time from the results of said motion analysis, and (2) automatically detecting respiratory events based on said airflow signal, wherein a respiratory event is a breathing disturbance; and
a report generator for producing at least one report about said respiratory events.

16. The system of claim 15 wherein said second network interface transmits at least one of said at least one report about said respiratory events across said network to a remote display device.

17. The system of claim 15 wherein said second computing device further comprises a state detector for analyzing said results of said motion analysis and for automatically inferring information about the state of the subject.

18. The system of claim 15 wherein said first computing device further comprises a monitor for displaying the received images.
